(19) United States
(12) Patent Application Publication
(10) Pub. No.: US 2025/0194570 A1
(43) Pub. Date: Jun. 19, 2025

(54) HYBRID MICROBEADS WITH BIODEGRADABLE SURFACE AND HETEROGENEOUS SIZE DISTRIBUTION, AND COMPOSITIONS AND METHODS THEREOF

(71) Applicant: University of Massachusetts, Westborough, MA (US)

(72) Inventors: Qianbin Wang, Endicott, NY (US); Zhigang He, Wellesley, MA (US); Siyuan Rao, Endicott, NY (US); Eunji Hong, Binghamton, NY (US)

(21) Appl. No.: 18/803,417

(22) Filed: Aug. 13, 2024

Related U.S. Application Data
(60) Provisional application No. 63/532,524, filed on Aug. 14, 2023.

(51) Int. Cl. A01K 67/027; A61L 24/00; A61L 24/04

(52) U.S. Cl. CPC A01K 67/027; A61L 24/001; A61L 24/043; A01K 2227/105; A01K 2267/035; A61L 2430/36

(57) ABSTRACT

The invention provides novel hybrid polystyrene (PS)/poly(lactic-co-glycolic acid) (PLGA) core-shell microbeads having viscous surface coating and heterogeneous size distribution, and methods of their preparation and use in animal models.

### PRIORITY CLAIMS AND RELATED PATENT APPLICATIONS

This application claims the benefit of priority to U.S. Provisional Application Ser. No. 63/532,524, filed on Aug. 14, 2023, the entire content of which is incorporated herein by reference in its entirety.

### TECHNICAL FIELD OF THE INVENTION

The invention generally relates to polymer and microscopic materials. More particularly, the invention relates to polystyrene (PS)/poly(lactic-co-glycolic acid) (PLGA) hybrid polymeric beads and their use in animal models and methods thereof.

### BACKGROUND OF THE INVENTION

Glaucoma is a progressive and irreversible blinding neuropathy that affects millions of people. The intraocular pressure (IOP) elevation is generally recognized as a major risk factor in glaucoma disease progression. The pathophysiological mechanisms of glaucomatous neurodegeneration remain poor understand due to the lack of reliable glaucoma animal models to recapitulate the features, including sustained IOP elevation and progressive retinal ganglion cells (RGCs) death.

Laboratory mouse models provide a useful mammalian system to study the pathophysiology of human diseases with various genetic approaches; however, current transgenic mouse models, such as DBA/2J, are limited by unstable IOP elevation, which leads to large variations in neural mechanism investigation. Various experimental glaucoma models have been established, including translimbal laser photocoagulation models, microbeads occlusion models, and a recently developed silicone oil occlusion model. Among these experimental glaucoma models, microbead occlusion models with uniform-sized PS or latex microspheres have gained popularity due to their relatively straightforward operations and rapid IOP elevation.

Polystyrene or latex microspheres with uniform size distribution have been used by researchers. Silicon oil has been utilized to create server and acute occlusion models and expanded to large animals, such as non-human primates. From a biophysics perspective, however, the rigidity of the polymeric beads and unmatched beads size to the trabecular meshwork (TM) heterogeneous structures can lead to inadequate stability of beads-tissue contact and unsatisfied beads retention in TM tissues. As a result, currently available microbead occlusion models require multiple injections to maintain long-term ocular hypertension.

In some strategies, viscous substances, such as hyaluronic acid (HA), are injected first followed by polymeric microbeads injection. The additional injection of viscous substances can induce a rapid IOP elevation in the first several days; however, due to the lack of mechanical support, such soft materials will degrade and clear out by metabolic processes, which hampers the long-term stability of occlusion.

There remains a significant unmet need for novel approaches to microbeads and animal models that overcome these limitations.

### SUMMARY OF THE INVENTION

The invention provides novel polymeric microbeads having biodegradable viscous surface coating and heterogeneous size distribution. These core-shell structured viscobeads are synthesized by combining non-degradable PS hard core with a biodegradable PLGA soft surface. In particular, the degraded PLGA surface became viscoelastic materials and glued the individual beads with tissue. The prolonged beads-tissue adhesion can be further achieved for a long-term blockage effect.

Another feature of viscobeads, which serves to ensure the effective blockage, is the size match between their heterogeneous size distribution and the gradient porous structures in the TM. The TM is made up of several distinct cell layers, including uveal meshwork (UM), corneoscleral meshwork (CM), the juxtacanalicular tissue (JCT), and the endothelial lining of Schlemm's canal (SC). The pore sizes of TM from initial UM to inner SC are distributed from about 25-75 μm to about 0.2-2 μm.

Results disclosed herein demonstrated that the PS/PLGA hybrid viscobeads with 1-15 μm size distribution can induce a consistent IOP elevation and progressive RGCs loss in about 8 weeks with a single injection

In one aspect, a bead comprising: a core comprising PS or latex; and a shell encapsulating the core, wherein the shell comprises PLGA, poly(glycolic acid) (PGA) or polylactic acid (PLA), wherein the core has a diameter in the range of about 1 μm to about 20 μm, and the shell has a thickness in the range of about 1 μm to about 10 μm.

In another aspect, the invention generally relates to a composition comprising a plurality of microbeads disclosed herein.

In yet another aspect, the invention generally relates to an animal glaucoma model induced by the plurality of beads disclosed herein.

In yet another aspect, the invention generally relates to a method for preparing an animal glaucoma model, comprising injecting to the anterior chamber of the animal's eyes a plurality of beads disclosed herein.

In yet another aspect, the invention generally relates to a method for preparing PS/PLGA core-shell beads, comprising: providing a first emulsion of PS in an organic solvent; proving a second aqueous solution of poly(vinyl alcohol) (PVA); mixing the first emulsion with the second solution to form a double emulsion; evaporating the organic solvent and removing the aqueous solvent to obtain PS microspheres; adding the PS microspheres to a PLGA solution, followed addition of the aqueous PVA solution and emulsification of the resulting solution; and isolating the prepared PS/PLGA microspheres.

In yet another aspect, the invention generally relates to a plurality of PS/PLGA core-shell beads prepared according to the method disclosed herein, wherein the PS core has a diameter in the range of about 1 μm to about 20 μm; the PLGA shell has a thickness in the range of about 1 μm to about 10 μm; and the plurality of beads is characterized by a heterogeneous size distribution of beads within the range of about 1 μm to about 50 μm.

### BRIEF DESCRIPTION OF THE DRAWINGS

FIG. 1: Bioinspired Design Injectable Viscobeads with Heterogeneous Size Distribution and Biodegradable Surface Properties. (A) Illustrations of the mouse eye, with the anterior segment comprising the cornea, iris, ciliary body, and lens. The ciliary body secretes aqueous humor, which circulates (blue arrows) within the anterior chamber before draining from the eye. (B) Enlarged view of the aqueous humor outflow pathway (blue arrows) in the iridocorneal angle. In the conventional pathway, the aqueous humor traverses the trabecular meshwork, first passing through the uveal meshwork (UM, highlighted in blue), then the corneoscleral meshwork (CM, highlighted in pink), and finally, the juxtacanalicular tissue (JCT, highlighted in yellow) before entering Schlemm canal. (C) An illustrative depiction of the injectable PS/PLGA viscobeads technique. Viscobeads were injected intracamerally into the iridocorneal angle of the eye, blocking the aqueous humor outflow pathway. (D) A representative fluorescence image revealing the accumulation of rhodamine B-labeled PS/PLGA viscobeads at the mouse iridocorneal angle. Scale bar: 100 μm. (E-F) Representative scanning electron microscopy (SEM) images indicating that the trabecular meshwork exhibits a gradient porous structure with a pore size range of approximately 1-20 μm. Scale bar: 10 μm. (G) A simplified model illustrating viscobeads blockage in the aqueous humor outflow pathway with three layers: UM, CM, and JCT. The initial interlaminar spaces in the UM measure approximately 25-27 μm, retaining Viscobeads with larger sizes. The smaller side of viscobeads tends to clog in the JCT layer, which has a size range of 0.2-2 μm, while the middle-sized particles become trapped in the CM layer with sizes ranging from 2 to 15 μm. Simulation of the physical blockage of aqueous humor outflow was conducted using ANSYS Fluent. Heterogeneously distributed particles (Gaussian distribution with sizes ranging from 1 to 15 μm) were released from the inlet of a three-layered structure with gradient gap sizes to mimic the UM, CM, and JCT layers (10, 5, and 2 μm from inlet to outlet, respectively) in a discrete phase model. Color lines indicate trajectories of particles with different sizes. (H) Representative SEM images indicating the entrapment of larger viscobeads on the surface of the initial interlaminar spaces of the trabecular meshwork. (J) A representative SEM image of PS/PLGA viscobeads displaying a size distribution ranging from 1 to 20 μm. Scale bar: 20 μm. (K-L) Size distribution of the initially administered PS/PLGA viscobeads with an average of 9.61±3.74 μm, and size distribution of the viscobeads entrapped on the surface of the initial interlaminar spaces of the trabecular meshwork with an average of 11.90±4.56 μm. (M) Size distribution of particles at different layers (inlet, P1, P2, and outlet) in the ANSYS Fluent model indicates particle entrapment in distinct layers. (N) Intraocular pressure (IOP) in mice before and after the injection of PS/PLGA viscobeads or the saline group. (O) The duration of elevated IOP in mice injected with 1-15 μm PS microbeads, 15 μm and 4.2 μm commercial microbeads, saline, and the naïve group. n=4 for 1-15 μm PS, 5 for other groups. Two-way measures ANOVA with Tukey's multiple comparisons test (F(60,285)=2.847, ****P<0.0001, F(6.829,129.7)=10.34, ****P<0.0001, F(4, 19)=81.58, ****P<0.0001, F(19,285)=1.674, P=0.0400 (P) A representative photograph of eyes extracted 8 weeks post-injection of viscobeads (glaucoma) and saline.

FIG. 2: The Mechanism of Self-Production of Viscoelastic Agents Through Degradation. (A) PLGA degrades into low molecular weight PLGA or monomers, which serve as viscoelastic materials. (B) PLGA is a linear copolymer composed of tunable constituent monomers, lactic acid (L) and glycolic acid (G). (C) Raman spectra of PLGA viscobeads before and after submersion for 7 days at 37° C. and 55° C. (D-E) The illustration demonstrates that the biodegradable PLGA acts as a viscous substance, efficiently binding the viscobeads to the surrounding tissue, thereby augmenting the entrapment of beads in TM tissue and contributing to effective aqueous humor blockage. (F-G) Representative SEM images show viscobeads immersed in a 55° C. water bath for 7 days in the in vitro condition (F) and viscobeads entrapped on the surface pores of the trabecular meshwork (G). The red arrow indicates the degraded PLGA viscous substance. Scale bar: 20 μm for (F) and 10 μm for (G). (H) The IOP during the first week in mice injected with different microbead compositions. Two-way measures ANOVA with Tukey's multiple comparisons test (F(9, 48)=22.62, ****P<0.0001, F(1.260, 20.16)=61.05, ****P<0.0001, F(3, 16)=51.94, ****P<0.0001, F(16, 48)=1.814, P=0.0570). (I-J) Different IOP levels (I) and durations of IOP elevation (J) in mice injected with saline, PLGA, PS, and PS/PLGA viscobeads containing 10%, 5%, and 3% of PLGA components (w/w). One-way measures ANOVA with Tukey's multiple comparisons test for I (F(5, 24)=26.76, ****P<0.0001) and J (F(2, 9)=21.78, P=0.0004).

FIG. 3: Glaucomatous Neurodegeneration in the Viscobeads-Induced Ocular Hypertension Model. (A) Representative wholemount retinal confocal images from mice at 0, 4, and 8 weeks after PS/PLGA viscobeads injection. RBPMS antibody was used for immunohistochemical staining of RGCs. Scale bars: 100 μm. (B) Quantification of RGC survival at different time points after PS/PLGA viscobeads injection. One-way measures ANOVA with Tukey's multiple comparisons test (F(2, 22)=39.98, ****P<0.0001). (C-E) Representative confocal images (C-D) and quantification (E) of retinal cryosections (C) and optic nerve head (D) stained with DAPI and IBA1 at days 0, 3, 5, and 7 post-injections. Scale bars: 50 μm. Two-way measures ANOVA with Sidak's multiple comparisons test (F(3, 24)=0.8545, P=0.4780, F(3, 24)=28.67, ****P<0.0001, F(1, 8)=3.026, P=0.1202, F(8, 24)=1.917, P=0.1040). (F-K) Electrophysiological assessments using pattern electroretinogram (pERG) and flash visual evoked potential (VEP) to evaluate RGC function and visual pathway integrity during glaucoma progression. (F) Representative pERG signals in mice from week 0 (pre-injection) to week 6 post-injection. (G) Representative confocal images of retinal sections. Scale bar: 50 μm. (H-I) Statistical quantification of the amplitudes of P1 and N1 of the pERG signals before and after viscobead injection. One-way repeated measures ANOVA with Tukey's multiple comparisons test (pERG-P1: F(3, 52)=42.44, ****p<0.0001; pERG-N2: F(3, 53)=65.68, ****p<0.0001). (J-K) Statistical quantification of the amplitude (J) and latency (K) of N1 of the VEP signals before and after viscobead injection. One-way repeated measures ANOVA with Tukey's multiple comparisons test (Amplitude: F(3, 60)=7.292, p=0.0003, 0 W vs. 6 W ***p=0.0001, 2 W vs. 6 W **p=0.0051, 4 W vs. 6 W *p=0.0271; Implicit time: F(3, 60)=25.83, ****p<0.0001, 0 W vs. 2 W, ***p=0.0005).

FIG. 4: Manipulating Genes in the Retina to Prevent RGC Loss in the Viscobeads-Induced Glaucoma Model. (A) Schematic illustration of AAV2 vectors injected intravitreally 2 weeks before microbeads injection. (B) IOP elevation in different groups of mice. (C) Representative images of retinal sections showing RGC survival with AAV injections. Scale bar: 20 μm. (D) Quantification of RGC survival after 8 weeks of PS/PLGA hybrid microbeads injection. (E) Representative confocal images of optic nerve cross-sections from mice with saline injection or non-targeting sgRNA, PTEN, and LIN28 AAV injection. Scale bar for the upper panel: 100 μm; bottom panels: 20 μm. One-way measures ANOVA with Dunnett's multiple comparison test. (Non-target gene vs. Other groups: F(8, 75)=21.26 ****p<0.0001, Saline ****p<0.0001, Pten ***p=0.0002, Lin28 ****p<0.0001, ATF3 **p=0.0019, Chop **p=0.0024).

FIG. 5: The schematical illustration of PLGA/PS Viscobeads fabrication process. To prepare the polystyrene (PS) microspheres, 1 mL of dye aqueous solution (optional) was added to 3 mL of 0.1 g/mL PS/chloroform, followed by emulsification using a high-speed homogenizer at 5000 rpm for 30 s. This primary emulsion was immediately poured into 15 mL of 2% PVA aqueous solution and stirred to form a double emulsion. The organic solvents were evaporated by pouring the mixture into 100 mL of 55° C. water and stirring for 30 minutes. The resulting PS microspheres were then washed with DI water three times and centrifuged at 2000 rpm. For surface coating, the dried PS microspheres were added to 3 mL of 0.02 g/mL Poly Lactic-co-Glycolic Acid (PLGA) in dichloromethane (DCM). This mixture was then emulsified using a high-speed at 5000 rpm for 30 s after adding 15 mL of 2% PVA aqueous solution. After the organic solvents were evaporated, PS/PLGA microspheres were centrifuged at 2000 rpm and washed with saline three times.

FIG. 6: Representative SEM images of PLGA/PS viscobeads before injection (A) and after 8 weeks of injection (B-C) suggest that degraded PLGA could function as a viscous substance, efficiently adhering individual microbeads in vivo. This is confirmed by SEM observations of the remaining beads on the iris surface.

FIG. 7: (A) The simulation of physical blockage in aqueous humor outflow was conducted using ANSYS Fluent. Heterogeneously distributed particles (Gaussian distribution with sizes ranging from 1 to 15 μm) were released from the inlet of a three-layered structure with gradient gap sizes (10, 5, and 2 μm from inlet to outlet, respectively) in a discrete phase model. The color lines indicate trajectories of particles with different sizes. (A1-A4) Enlarged views show the outflow of the fluid with particles sized 1-15 μm at different layers (inlet A1, P1: A2, P2: A3, and outlet: A4), (B) The size distribution of particles at different layers (inlet, P1, P2, and outlet) in the ANSYS Fluent model indicates particle entrapment in distinct layers. (C) Normalized pressure changes at different positions from the inlet to the outlet.

FIG. 8: The schematic illustration of PLGA/PS hybrid microbeads injection process. A. (1) Aqueous humor normally flows from the ciliary body into the anterior chamber and then into a drainage canal in a healthy eye. (2) In the glaucoma injection procedure, a bubble is initially introduced into the anterior chamber to prevent potential leakage. (3) Subsequently, a 1 uL solution containing PS/PLGA hybrid microbeads is injected into the anterior chamber. (4) The PS/PLGA hybrid microbeads aggregate at the anterior chamber angle, obstructing aqueous drainage and leading to an increase in intraocular pressure (IOP). B. Representative photographs of a mouse eye taken before and after injection. The white PS/PLGA hybrid microbeads are injected into the anterior chamber (middle) and become accumulated at the iridocorneal angle within 5 minutes after injection (bottom).

FIG. 9: The duration of elevated IOP in mice injected with saline and the PS/PLGA Viscobeads. n=5 for saline group and n=10 for Viscobeads group.

FIG. 10: Intraocular pressure in mice injected with 1-15 μm PS microbeads, 15 μm and 4.2 μm PS microbeads, saline, and the naïve group. n=4 for 1-15 μm PS, 5 for other groups.

### DETAILED DESCRIPTION OF THE INVENTION

The invention is based in part on the novel approach based on PS/PLGA hybrid core-shell beads that exhibit a viscous surface coating and heterogeneous size distribution.

In contrast to the rigid and non-degradable polymeric beads, core-shell structured viscobeads disclosed herein employ a non-degradable PS hard core encapsulated with a biodegradable PLGA shell with a soft surface. The degraded PLGA surface becomes viscoelastic and glues the individual beads to the tissue. The prolonged beads-tissue adhesion enables a long-term blockage effect.

To further ensure effective blockage, the viscobeads exhibits a heterogeneous size distribution that matches the gradient porous structures in the TM. The TM is made up of several distinct cell layers, including UM, CM, JCT, and the endothelial lining of SC. The pore sizes of TM from initial UM to inner SC are distributed from 25-75 μm to 0.2-2 μm.

In one aspect, a bead comprising: a core comprising PS or latex; and a shell encapsulating the core, wherein the shell comprises PLGA or PGA or PLA, wherein the core has a diameter in the range of about 1 μm to about 20 μm (e.g., about 1 μm to about 15 μm, about 1 μm to about 10 μm, about 1 μm to about 5 μm, about 5 μm to about 20 μm, about 10 μm to about 20 μm, about 5 μm to about 15 μm), and the shell has a thickness in the range of about 1 μm to about 10 μm (e.g., about 1 μm to about 7 μm, about 1 μm to about 5 μm, about 1 μm to about 3 μm, about 2 μm to about 10 μm, about 5 μm to about 10 μm).

In certain embodiments of the bead, the core is non-degradable. In certain embodiments of the bead, the core is degradable.

In certain embodiments of the bead, the core is PS.

In certain embodiments of the bead, the shell is PLGA.

In certain embodiments of the bead, the PS of the core is characterized by a MWₙ of about 5,000 to about 400,000 (e.g., about 5,000 to about 250,000, about 5,000 to about 100,000, about 5,000 to about 50,000, about 5,000 to about 20,000, about 10,000 to about 400,000, about 20,000 to about 400,000, about 50,000 to about 400,000, about 10,000 to about 100,000); and the PLGA of the shell is characterized by a MWₙ of about 1,000 to about 300,000 (e.g., about 1,000 to about 150,000, about 1,000 to about 100,000, about 1,000 to about 50,000, about 1,000 to about 10,000, about 5,000 to about 300,000, about 10,000 to about 300,000, about 50,000 to about 300,000, about 10,000 to about 100,000). In certain embodiments the PS is characterized by a MWₙ of about 35,000 to about 50,000; and the PLGA is characterized by a MWₙ of about 25,000 to about 35,000.

Any suitable ratios of LA vs GA may be employed. In certain embodiments, the PLGA has about 5% to about 95% (e.g., about 10% to about 90%, about 15% to about 85%, about 20% to about 80%, about 25% to about 75%) lactic acid (LA) and about 95% to about 5% (e.g., about 90% to about 10%, about 85% to about 15%, about 80% to about 20%, about 75% to about 25%) glycolic acid (GA). In certain embodiments, the PLGA has about 35% to about 65% LA and about 65% to about 35% GA.

In certain embodiments, the core has a diameter in the range of about 1 μm to about 10 μm, and the shell has a thickness in the range of about 1 μm to about 10 μm.

In certain embodiments of the bead, the core is latex.

In certain embodiments of the bead, the shell is PGA.

In another aspect, the invention generally relates to a composition comprising a plurality of microbeads disclosed herein.

In certain embodiments, the plurality of beads is characterized by a heterogeneous size distribution of beads within the range of about 1 μm to about 50 μm (e.g., about 1 μm to about 40 μm, about 1 μm to about 30 μm, about 1 μm to about 20 μm, about 5 μm to about 50 μm, about 10 μm to about 50 μm).

In certain embodiments, the size distribution is in the range of about 1 μm to about 20 μm (e.g., about 1 μm to about 15 μm, about 1 μm to about 10 μm, about 5 μm to about 20 μm, about 5 μm to about 15 μm).

In yet another aspect, the invention generally relates to an animal glaucoma model induced by the plurality of beads disclosed herein.

In certain embodiments, the animal is a mouse.

In certain embodiments, the animal model is characterized by an IOP elevation of about 5 mm Hg to about 25 mm Hg (e.g., about 5 mm Hg to about 20 mm Hg, about 10 mm Hg to about 25 mm Hg, about 15 mm Hg to about 25 mm Hg).

In certain embodiments, the IOP elevation is about 10 mm Hg to about 20 mm Hg.

In yet another aspect, the invention generally relates to a method for preparing an animal glaucoma model, comprising injecting to the anterior chamber of the animal's eyes a plurality of beads disclosed herein.

In certain embodiments of the method, the animal is a mouse.

In yet another aspect, the invention generally relates to a method for preparing PS/PLGA core-shell beads, comprising: providing a first emulsion of PS in an organic solvent; proving a second aqueous solution of PVA; mixing the first emulsion with the second solution to form a double emulsion; evaporating the organic solvent and removing the aqueous solvent to obtain PS microspheres; adding the PS microspheres to a PLGA solution, followed addition of the aqueous PVA solution and emulsification of the resulting solution; and isolating the prepared PS/PLGA microspheres.

In certain embodiments of the method, the organic solvent is chloroform.

In certain embodiments of the method, the PLGA solution is PLGA in dichloromethane.

In yet another aspect, the invention generally relates to a plurality of PS/PLGA core-shell beads prepared according to the method disclosed herein, wherein the PS core has a diameter in the range of about 1 μm to about 20 μm; the PLGA shell has a thickness in the range of about 1 μm to about 10 μm; and the plurality of beads is characterized by a heterogeneous size distribution of beads within the range of about 1 μm to about 50 μm.

The viscobeads technology described provides a bio-interface solution to manipulate fluidic transport in heterogeneously structured biological tissues and can be directly applied as an injectable technique to create an experimental glaucoma mouse model by sustained intraocular hypertension. The “hard” polymer core provides mechanical support for long-term retention while the biodegradable “soft” surface provides viscoelastic contact to tissues, yielding composite viscoelastic particles. Tuning the composition of the hybrid materials can affect the surface materials' biodegradation dynamics and further regulate the occlusion effect. Instead of using uniform-sized microbeads, the viscobeads (1-20 μm) were designed to match the size gradient of porous structures in the trabecular meshwork that regulate intraocular pressure. The combination of hybrid material composition and a precise size distribution optimized the beads-tissue infiltration after a single-dose and produced long-term bead retention in the TM. IOP elevation (21.4±1.39 mm Hg) was subsequently sustained over 8 weeks, corresponding to observed glaucomatous neurodegeneration. Over 34% RGC death was noted along with a dramatic oscillatory potential change in electroretinography. While analyzing pERG and VEP signals and retinal tissues, we found that RGC function is impaired at an early stage of ocular hypertension, even before observable RGC loss in tissues. In the later stages of glaucoma, the integrity of the visual pathway is likely affected in this experimental model.

The viscobeads design roots in the in vivo AH fluid flow dynamics in the TM architectures. Instead of creating a surface occlusion with uniform-sized beads, heterogeneously sized beads, which match the TM porous structures, create a bulk occlusion effect among different layers of TM tissues. Viscoelastic surfaces of the hybrid beads further offer prolonged retention under complex fluid flow conditions. The size distribution and biodegradation dynamics of viscobeads can be directly tuned from synthesis, which supports a convenient engineering approach to adapt the viscobeads occlusion model to other tissue structures.

The viscobeads experimental glaucoma model provides a reliable in vivo testbed that replicates glaucoma-like high IOP and retinal degeneration. Introducing the viscobeads is compatible with other surgical procedures, such as injecting viral vectors to manipulate gene expression. In a proof-of-concept application, we used the viscobeads glaucoma model to assay a series of previously selected gene candidates and gained new insights into the neuroprotective effects of these genes. Notably, during this screening, we identified that LIN28 exhibited detrimental effects on RGC survival. Our findings challenge the widely held belief that the optic nerve crush model sufficiently mimics glaucoma neurodegeneration. The distinct results for LIN28 between the optic nerve crush model⁵¹ and our experimental glaucoma model underscore our model's reliability and its potential to provide more accurate insights into glaucoma pathology.

The viscobeads technique offers a new perspective on the materials-tissue interfaces used to regulate fluid transport in vivo. Similar interface designs may be further extended to other biological tissues that regulate bio-fluid flow with micro-architectures, such as the lymphatic system, pulmonary alveolus, and bones. As a tool to study the neurodegeneration of glaucoma, the viscobeads technique provides a scalable and accessible engineering method to introduce in vivo occlusion for models of ocular hypertension. The injection is minimally invasive and compatible with the delivery of other genetic or pharmaceutical reagents. By sustaining a prolonged and stable high IOP in vivo condition upon a single injection, the viscobeads technique provides a reliable testbed for future therapeutic intervention development.

### EXAMPLES

The Examples below describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### Design of Viscobeads Matching the Sizes of Trabecular Meshwork Fenestrations

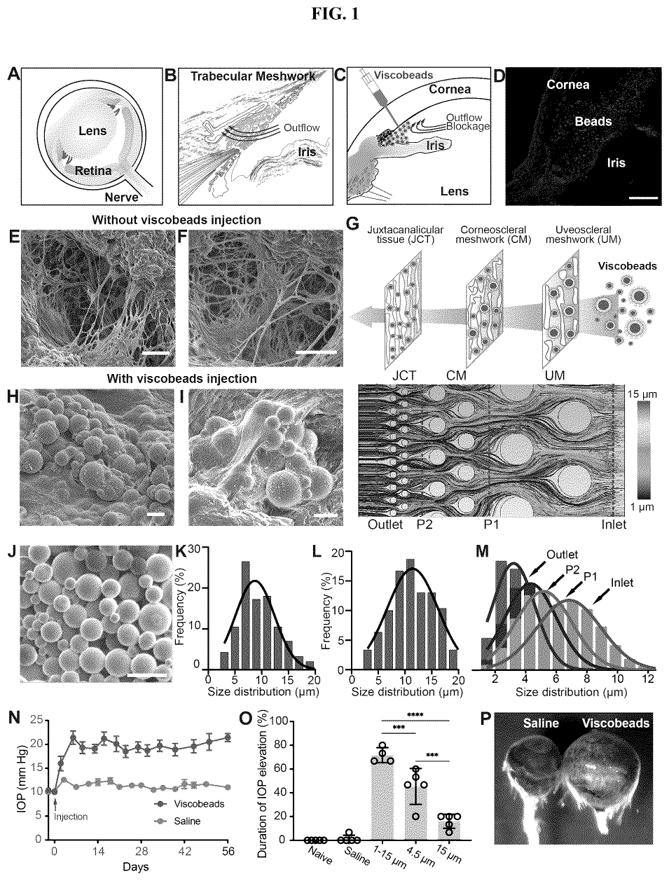

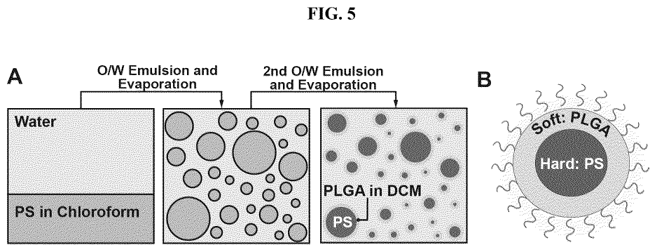

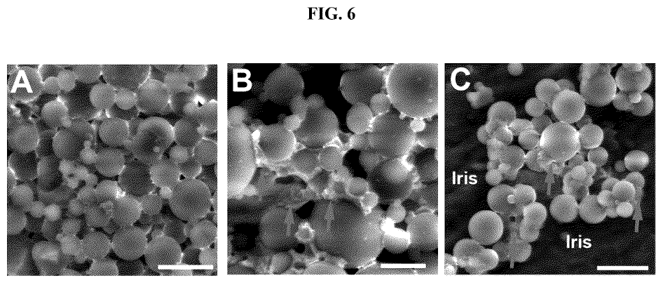

We synthesized PS/PLGA viscobeads with a controllable size distribution using the single-emulsion method (FIG. 5). These beads feature a robust core and a viscoelastic surface, attributes crucial for prolonging retention in the TM. Hydrophobic PS functions as a rigid skeletal structure to ensure long-term stability, while hydrophilic and biodegradable PLGA provides viscoelastic surfaces after degradation in vivo (FIG. 6). Properly tuning the size distribution of the viscobeads is essential to align with the porous structure of the trabecular meshwork (TM) to improve the occlusive effect on the drainage of AH at the iridocorneal angle, and consequently induce and maintain prolonged IOP elevation. In the eye, the ciliary body secretes AH, which circulates within the anterior chamber before draining from the eye through the TM. In the conventional AH outflow pathway, the AH traverses the TM, initially passing through the uveal meshwork (UM) through the corneoscleral meshwork (CM) and finally to the juxtacanalicular tissue (JCT) before entering Schlemm's canal (FIG. 1A-B). Upon intracameral injection of microbeads into anesthetized mice, microbeads rapidly accumulated at the iridocorneal angle, were effectively retained across the structurally graded sizes of TM pores, and blocked AH drainage (FIG. 1C-D).

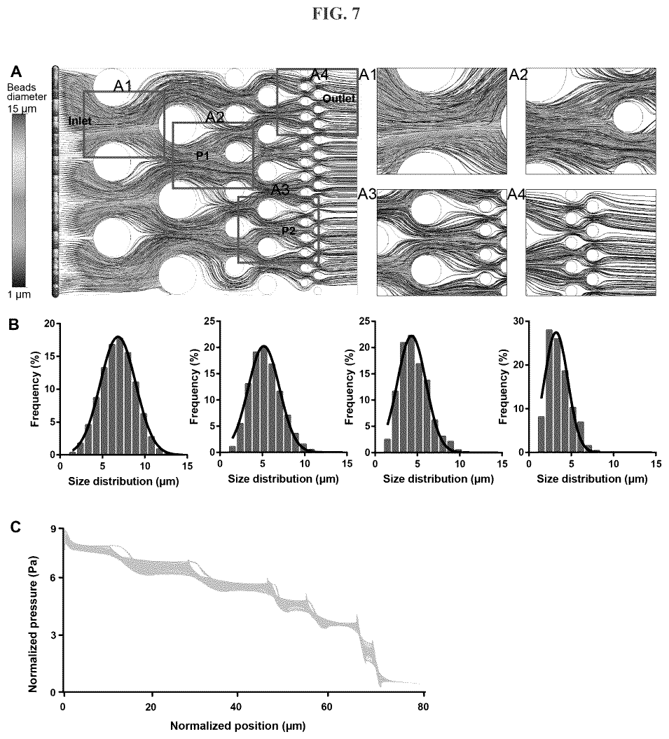

To depict viscobeads occlusion in the AH outflow pathway, we developed a simplified model featuring three layers-UM, CM and JCT—each characterized by pore sizes falling within a 1-20 μm range (FIG. 1E-DF). The UM's initial interlaminar spaces measure approximately 25-27 μm, accommodating beads with larger sizes. Smaller beads tend to clog in the JCT layer, which has a size range of 0.2-2 μm, while middle-sized particles become trapped in the CM layer with sizes ranging from 2 to 15 μm. To mimic the natural size progression of TM structures, we established a discrete phase model (ANSYS Fluent simulation, FIG. 1G, and FIG. 7) and simulated the blocking effect of size-mixed spheres flowing through different layers. Heterogeneously distributed particles (Gaussian distribution with sizes ranging from 1 to 15 μm) were released from the inlet of a three-layered structure with gradient gap sizes to mimic the UM, CM, and JCT layers (10, 5, and 2 μm from inlet to outlet, respectively) in a discrete phase model. The blocking effect is visualized with the size distribution of the trapped spheres and generally characterized into four planes: the inlet where the spheres were released, P1 (the boundary between larger and medium gaps), P2 (the boundary between medium and small gaps), and the outlet. The average sphere size changes from 7 μm at the inlet to 3.3 μm at the outlet, indicating that the majority of the spheres were retained in the layered structures. We also observed the entrapment of larger beads on the surface of the initial interlaminar spaces of the trabecular meshwork after 8-week post viscobeads injection (FIG. 1H-I).

By precisely adjusting the power, duty cycle, and ultrasound time during the oil-in-water synthesis processes, we achieved a size range of 1-20 μm for viscobeads, with an average size of 9.61±3.74 μm (FIG. 1J-K). In the context of a gradient porous structure, particles of different sizes are trapped in alignment with the local pore size. Larger pores accommodate beads with larger sizes, smaller beads tend to infiltrate layers with small pores, and middle-sized particles become trapped in intermediate pore structures. This phenomenon is supported by both experimental SEM observations and theoretical modeling in the ANSYS Fluent simulation model (FIG. 1L-M). In the 8 weeks following viscobeads injection, beads with an average size of 11.90±4.56 μm were observed to be effectively trapped on the trabecular meshwork pores (FIG. 1L).

### Effect of Viscobeads Size Distribution on IOP Elevation

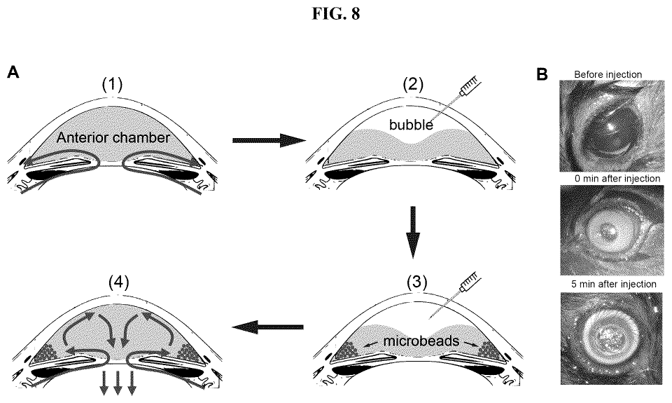

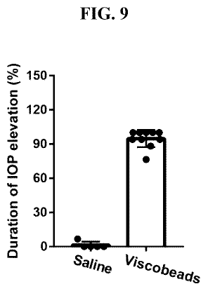

To confirm the effect of size distribution on AH drainage in vivo, heterogeneously sized viscobeads were injected into the eye (FIG. 8). Viscobeads instantly obstructed the porous structure of the trabecular meshwork, causing a rapid increase in IOP measured with tonometry. The IOP gradually rose to 21.4±1.39 mm Hg by the 7th-day post-injection and remained at elevated pressures at approximately 20 mm Hg for eight weeks. In contrast, the saline injection control group only achieved an IOP of 11.10±0.25 mm Hg (FIG. 1N, and FIG. 9).

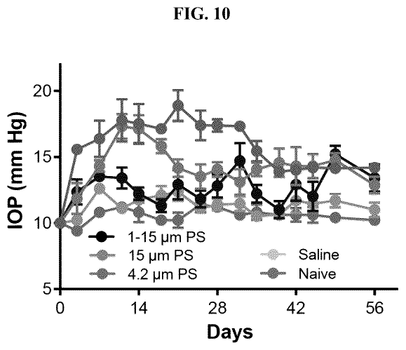

In the complex and dynamic fluidic environment of the AH, the transport of fluidic particles within the gradient porous structure of the trabecular meshwork is directly influenced by size distribution. To further assess the size effect of the microbead occlusion models, we compared viscobeads to commercially available microbeads. To eliminate any confounding effects of the viscoelastic surfaces, we synthesized pure PS microbeads with the same size distribution (1-20 μm) and compared the resulting IOP elevation in four groups of mice injected with commercial microbeads (4 μm or 15 μm, mainly composed of PS), saline solution, and no injection. IOP monitoring over eight weeks revealed that uniform-sized commercial beads, whether 4 μm or 15 μm, exhibited lower IOP elevation (14.40±0.26 mm Hg) and less stability compared to heterogenous-size PS beads (19.08±0.34 mm Hg) (FIG. 10). To better quantify IOP elevation over time, we defined a high IOP condition as persistently above 15 mm Hg for at least three consecutive days. Over the 8-week monitoring period, the duration of high IOP in the 1-15 μm PS microbeads injection group was 45.3±6.8%, significantly higher than in the commercial bead groups of 4 μm or 15 μm (p<0.0001) (FIG. 10). Due to the elevated pressure, mouse eyes injected with viscobeads displayed larger dimensions compared to eyes injected with saline at 8 weeks post-injection (FIG. 1P).

### Biodegradable Surface Design for Chronic IOP Elevation

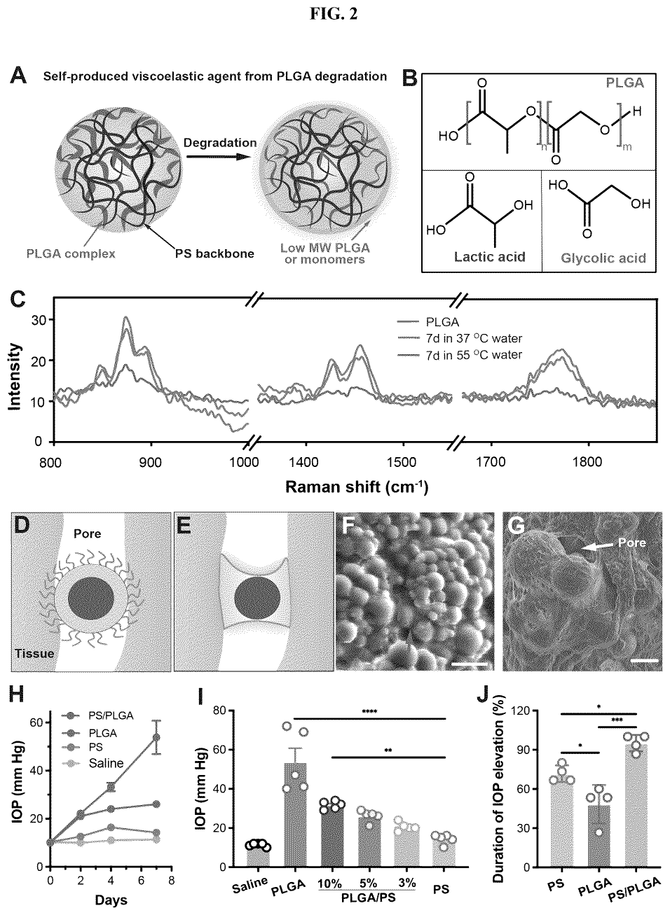

To improve the efficiency of AH flow blockage, we devised a strategy involving the incorporation of a biodegradable PLGA surface on the PS sphere cores. This design aimed to sustain elevated IOP by optimizing the retention of viscobeads in TM tissues. In contrast to previously reported methods involving sequential injections of viscous substances followed by rigid polymer microbeads, our approach leverages in vivo metabolic processes to degrade the surface PLGA materials, creating a viscoelastic contact between microbeads and tissues (FIG. 2A). To mimic in vivo degradation conditions, we initially examined the degradation dynamics of PLGA in vitro. PLGA, a linear copolymer with adjustable constituent monomers, lactic (L) and glycolic (G) acid⁴¹ (FIG. 2B), underwent incubation at 37° C. and 55° C. as an accelerated condition for 7 days. We measured the Raman spectra of PLGA glycolic and lactic units by comparing the intensities of featured peaks at 1452 and 1422 cm⁻¹ (FIG. 2C). In our design, the biodegradable PLGA serves as a viscous substance to promote adherence of the viscobeads to the surrounding tissue, effectively enhancing accumulation of the beads in the TM tissue and improving aqueous humor obstruction (FIG. 2D-E). Through SEM observations, we confirmed that the microbeads became connected after incubating under in vitro conditions (FIG. 2F, 7-day incubation at 55° C.) and adhering to the surrounding tissues (FIG. 2G, 56 days post-injection in vivo).

Manipulating microbeads composition can create distinct mechanical properties, and subsequentially impact IOP elevation dynamics in vivo. We prepared microbeads with a similar size distribution but different polymer compositions: pure PS, pure PLGA, and PS/PLGA viscobeads. Three groups of mice were injected with PS, PLGA and PS/PLGA viscobeads into the anterior chambers of mice, respectively, while the control group of mice were injected with the same volume of saline. Daily IOP measurements were taken in all groups over a 7-day post-injection period. Following the rapid degradation of PLGA, both the groups of mice injected with pure PLGA microbeads and PS/PLGA viscobeads exhibited a swift elevation in IOP two days post-injection (FIG. 2H). Due to the ongoing degradation of PLGA and the generation of viscous substances, the pure PLGA beads showed a dramatic increase in IOP to 53.8±6.93 mm Hg within one week, but the duration of maintaining high IOP was notably lower than PS or PS/PLGA viscobeads group (FIG. 2J). The IOP elevation in the pure PS group was much lower than that in the PLGA and PS/PLGA viscobeads groups. Mice injected with PS beads experienced a slight elevation (16.4±2.07 mm Hg on day 4), which eventually dropped to levels comparable to the saline-injected control group by the end of the 1-week monitoring period (14.2±1.11 mm Hg). Unlike the acute and severe blocking effect of pure PLGA microbeads or the insufficient blocking effect of pure PS microbeads, the PS/PLGA viscobeads group exhibited sufficient IOP elevation while maintaining it in a relatively stable high IOP range over time (26.0±1.34 mm Hg on day 7).

The elevated and stable IOP characteristic of the PS/PLGA hybrid viscobeads can be finely adjusted by manipulating the PLGA mass ratios because of the in vivo degradation dynamics of PLGA. We synthesized a series of PS/PLGA viscobeads with PLGA mass ratios of 3%, 5%, and 10%, resulting in varying IOP levels within the range of 20.6 mm Hg to 31.6 mm Hg (FIG. 2I). Under the conditions of pure PS or pure PLGA, the IOP exhibited either insufficient elevation or duration. Pure PS beads group reached 14.2 mm Hg, which was only 2.8 mm Hg higher than the saline group (11.4 mm Hg). Conversely, the pure PLGA beads achieved a severe acute elevation to 53.8 mm Hg but could sustain elevation for 48.3% of the observation period (FIG. 2I-J). Only the PS/PLGA viscobeads demonstrated both effective (>15 mm Hg) IOP elevation and maintaining 95% duration in the range of high IOP.

### Chronic High IOP-Induced Neurodegeneration

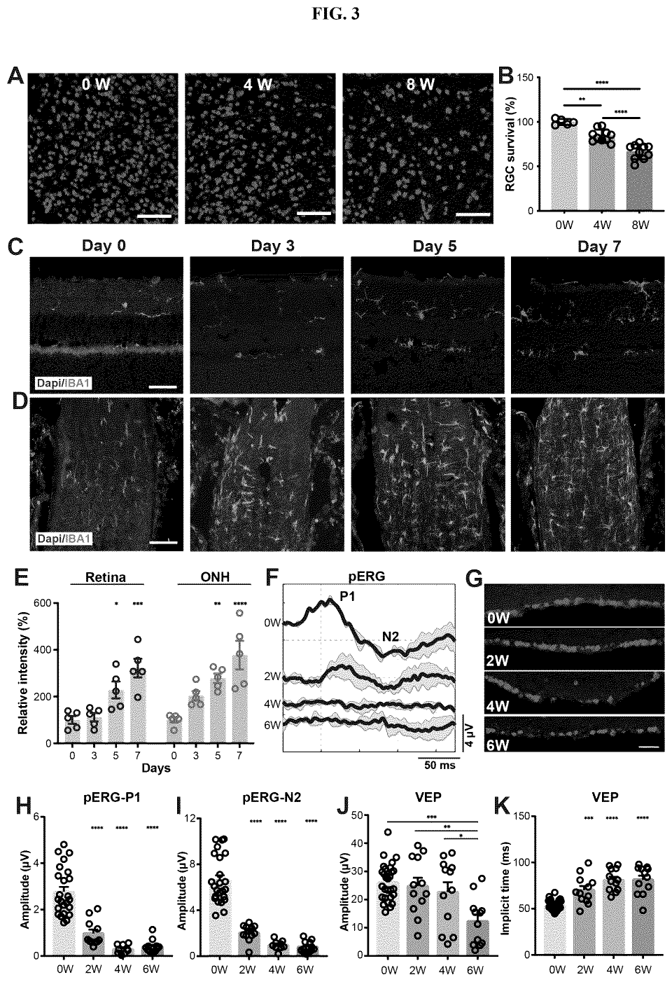

We assessed the chronic ocular hypertensive response and glaucomatous neurodegeneration by examining post-mortem retina tissues from mice injected with viscobeads. To evaluate the progression of glaucomatous neurodegeneration, we quantified the RGC survival rate at different time points post viscobeads injection. The somatic structure of surviving RGCs was labeled with RNA-binding protein with multiple splicing (RBPMS) antibodies on retinal wholemounts (FIG. 3A). To elucidate the impact of sustained high IOP on RGC death, we calculated the percentages of RBPMS-labeled RGCs from retinal tissues at a series of specified time points and compared them to retinal tissues dissected on the same day post injection (FIG. 3B). Notably, we observed a significant decrease in RGC survival percentages over an 8-week observation period. Among the eye tissues injected with viscobeads, the RGC survival rate decreased to 84.48±2.22% at week 4 and further declined to 66.43±2.63% at 8 weeks post-injection.

As an early event in the progression of glaucoma, microglia undergo morphological changes, proliferation, and migration into the injury site. Microglial cells produce proinflammatory cytokines and increase oxidative and nitrification reactions, exerting further negative effects on retinal neurons. To investigate the spatial and temporal dynamics of activated microglia during glaucomatous progression, we examined microglia and macrophages by immunolabeling the retina and optic nerve head (ONH) using ionized calcium-binding adaptor molecule 1 (IBA1) antibody (FIG. 3C-D). Concurrent with the chronic high IOP induced by the viscobeads, the intensity of IBA1+ cells in retinal and ONH tissues increased on day 3 post-injection, exhibited a dramatic rise on day 5, and further increased to over three times the baseline on day 7 (FIG. 3E).

### Electrophysiological Assessments of Changes in RGC Function and Visual Pathway Integrity During Glaucoma Progression

To monitor retinal function post-viscobead injection, we employed non-invasive pattern electroretinogram (pERG). pERG is a crucial electrophysiological assessment of general RGC function (FIG. 3F-I). In this test, the ERG responses were stimulated by contrast-reversing horizontal bars that alternate at a constant mean luminance. pERG measurements included the mean amplitude of the P1 and N2 components. The P1 component represents the initial positive deflection, originating from both RGCs and outer retinal photoreceptor cells. The mean P1 amplitude was measured as the difference between the average nadir of the N1 component and the average peak of the P1 component. The N2 component is the negative deflection that follows the P1 component, originating from the inner retina and reflecting RGC function. The mean N2 amplitude was calculated as the difference between the average peak of the P1 component and the average nadir of the N2 component (FIG. 3F). At week 2 post-viscobead injection, both the P1 and N2 components were significantly decreased compared to the control group with PBS injection, from 2.77±0.21 μV to 0.97±0.16 μV for P1 and from 6.62±0.42 μV to 2.04±0.20 μV for N2. This decrease continued to 0.40±0.08 μV for P1 and 0.80±0.14 μV for N2 at week 6 post-viscobead injection (FIG. 3H-I). These results suggest that RGC function diminishes along with glaucoma progression, even at an early stage where RGCs do not show a significant decrease based on RBMPS staining (FIG. 3G). It should also be noted that the injection of viscobeads may affect light intensity delivery to the retina, which can ultimately affect the pERG signal, indicating limitations in detection by pERG.

We then used flash visual evoked potential (VEP) to assess the functional retina and cortex as well as the state of visual pathways from the retina to the cortex. In VEP measurement, the electrophysiological signal is generated in the striate and extrastriate cortex. VEP measurements included the mean N1 amplitude and implicit time. The average N1 amplitude in the control group was 26.25±1.25 μV. In mice at 2, 4, and 6 weeks post-viscobead injection, the average N1 amplitudes were 24.83±2.95 μV, 22.69±3.47 μV, and 12.26±2.45 μV, respectively (FIG. 3J). The average implicit time of N1 in the control group was 53.84±1.05 ms, whereas the implicit time in mice at 2, 4, and 6 weeks post-viscobead injection were 70.17±4.39 ms, 81.21±3.47 ms, and 81.29±4.86 ms, respectively (FIG. 3K). The N1 implicit time values showed a significant increase compared to the control group with PBS injection. These results suggest that the integrity of the visual pathway is likely compromised in the later stages of glaucoma progression.

### A Viscobeads-Enabled Experimental Glaucoma Model for Gene Therapy Development

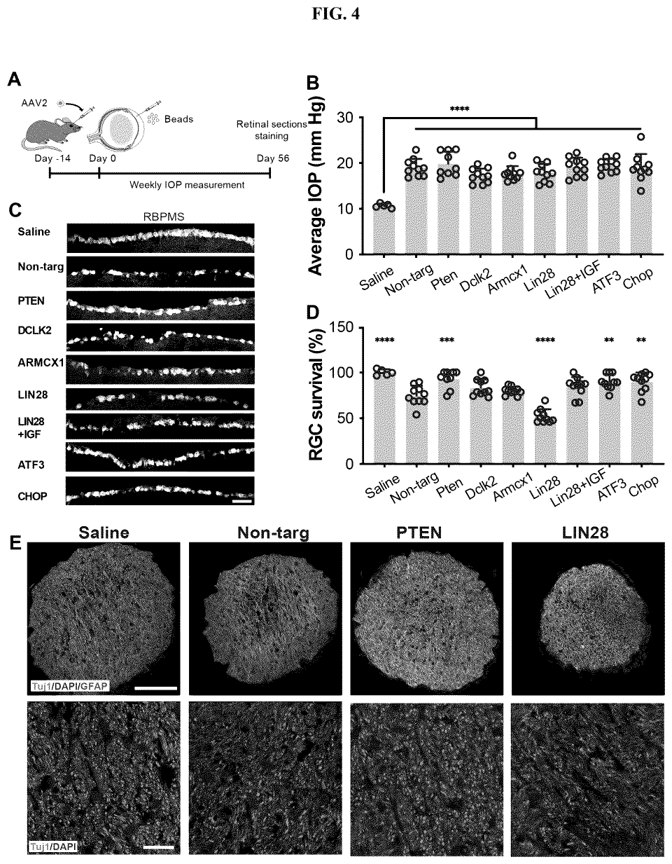

The reliability of sustaining chronic high IOP elevation and neurodegeneration supports a dependable and accessible model for the testing of therapeutic candidates. As a proof-of-concept for gene therapy development, we applied the viscobeads experimental glaucoma mouse model and tested genetic approaches to mitigate glaucomatous neurodegeneration. Gene candidates were initially selected based on their demonstrated neuroprotective effects in other axotomy animal models, such as optic nerve crush, as established in our prior research and by others. We chose to test phosphatase and tensin homolog (PTEN), a tumor-suppressor; activating transcription factor 3 (ATF3), a transcription factor; C/EBP homologous protein (CHOP), a transcription factor responsive to endoplasmic reticulum (ER) stress; doublecortin-like kinase 2 (DCLK2), a positive regulator involved in growth cone formation and axon regeneration; armadillo repeat-containing X-linked protein 1 (ARMCX1), a regulator of mitochondrial transport; Lin-28 homologue A (LIN28), an RNA-binding protein; and LIN28±IGF (insulin-like growth factor 1). Candidate genes or single-guide RNAs (sgRNA) were packaged into adeno-associated viruses (AAV) and introduced into the vitreous bodies of mouse eyes two weeks before intracameral injection of PLGA/PS viscobeads (day −14, FIG. 4A).

Over an 8-week observation period, all mice consistently exhibited elevated IOP (FIG. 4B), validating the reliability of viscobeads as an experimental glaucoma model and its compatibility with viral vector introduction. Among the selected gene candidates, we observed enhanced RGC survival rates in mice with PTEN, ATF3, and CHOP deletion. However, mice expressing LIN28 showed a dramatic decrease in survival rates after 8 weeks of viscobeads injection. The RGC survival rates of mice expressing ARMCX1, DCLK2, and LIN28±IGF showed no significant difference compared to the control group that received non-targeting sgRNA injections (FIG. 4C-D). Upon examining retinal sections with immunohistology stainings of RGC axons and glial cells, we identified similar protective effects of PTEN, ATF3, and CHOP, as well as an adverse effect of LIN28 on RGC axon density through immunohistochemistry of optic nerve cross-sections (FIG. 4E).

### PS/PLGA Viscobeads Fabrication

PS/PLGA viscobeads were prepared using a single emulsion method in an oil-in-water configuration. To prepare the polystyrene (PS) microspheres, 1 mL of rhodamine B (Sigma) aqueous solution (optional) was added to 3 mL of a 0.1 g/mL PS/chloroform solution. This mixture was then emulsified using a high-speed homogenizer at 5000 rpm for 30 seconds. The resulting primary emulsion was promptly poured into 15 mL of a 2% PVA aqueous solution and stirred to create a double emulsion. To remove the organic solvents, the mixture was transferred into 100 mL of 55° C. water and stirred for 30 minutes. The resulting PS microspheres were subsequently washed with deionized (DI) water three times and centrifuged at 2000 rpm. For surface coating, the dried PS microspheres were introduced into 3 mL of a 0.02 g/mL Poly PLGA solution in dichloromethane (DCM). After adding 15 mL of a 2% PVA aqueous solution, the mixture was emulsified at 5000 rpm for 30 seconds. Following the evaporation of organic solvents, PS/PLGA viscobeads were centrifuged at 2000 rpm, washed with saline three times, and then filtered using a 40 μL cell strainer.

### In Vitro Degradation and Characterization

To analyze the degradation dynamics of PS/PLGA viscobeads, Raman spectroscopy was conducted using a Thermo Fisher Scientific DXR2xi Raman microscope equipped with a 780-nm laser and a 10× microscope objective. PS/PLGA viscobeads were incubated for 7 days in different temperatures at 37° C., and 55° C., respectively. The viscobeads were then dried on a glass surface. Each spectrum was scanned from 3200 to 500 cm⁻¹ with 2 mW laser power and a 25-μm slit width for 5 seconds integration time. Eight scans were done automatically from different spots on the surface and then averaged by the instrument before analysis. To visualize the beads morphology, scanning electron microscopy was operated with dried samples using field-emission scanning electron microscopes (JEOL, JSM-6700F, Japan and Hitachi SU5000, Tokyo, Japan) under high vacuum conditions at 5 kV and 196-198 μA emission current, maintaining a working distance of 8.16-8.69 mm.

### Modeling

Fluid simulations were conducted by employing a discrete phase model within the ANSYS Fluent simulation software. The simulation geometry comprised three layers of pillar arrays with varying distances (10, 5, and 2 μm from inlet to outlet, respectively) to replicate the gradient porous structures found in the TM tissue. Size-mixed particles ranging from 1 to 15 μm were introduced into different layers to assess their blocking effects. To visualize this blocking phenomenon, we monitored the size distribution of particle flow at four distinct planes: the inlet where the particles were initially released, P1 (marking the boundary between larger and medium gaps), P2 (signifying the boundary between medium and small gaps), and finally, the outlet.

### Animals

C57BL/6J wild-type mice, obtained from Jackson Laboratory (JAX), aged between 6 to 8 weeks and weighing 20 to 25 grams were used for this study following the approved animal protocol by the Institutional Animal Care and Use Committee (IACUC) at Binghamton University and University of Massachusetts Amherst and complied with relevant regulations. All mice were housed in cages with a maximum of 5 mice per cage and subjected to a 12 h light/dark cycle, with unrestricted access to food and water.

### Intracameral Injection of Viscobeads

The elevation of IOP was induced by injecting viscobeads into the anterior chamber of the mouse eyes. The surgical procedures were adapted from a well-established microbead occlusion model and our pervious published work. In brief, anesthetized mice had their corneas gently punctured near the center using a 33 g needle (CAD4113, Sigma). A bubble was then introduced through this incision site into the anterior chamber to prevent possible leakage. Subsequently, 1 μL of viscobeads was injected into the anterior chamber. After a 5-minute interval during which the viscobeads accumulated at the iridocorneal angle, the mice were treated with antibiotic Vetropolycin ointment (Dechra Veterinary Products, Overland Park, KS) and placed on a heating pad for recovery.

### Intravitreal Injection of AAV

AAV vectors containing various gene candidates were delivered via intravitreal injection into mice eyes. Briefly, a pulled-glass micropipette was inserted near peripheral retina behind the ora serrata and deliberately angled to avoid damage to the lens. 2 ul of the AAV virus were injected into mice vitreous. Following the injection, we applied antibiotic ophthalmic ointment to the corneal surface.

### Intraocular Pressure Measurement

IOP measurements were conducted using a TonoLab tonometer (Colonial Medical Supply, Espoo, Finland) following the manufacturer's instructions. Prior to the measurements, the mice were anesthetized with a continuous flow of isoflurane (3% isoflurane in 100% oxygen). The average IOP was automatically calculated based on five measurements, after excluding the highest and lowest values.

Pattern Electroretinogram (pERG) Recording

Pattern ERG recording was performed on mice before surgery, 2, 4, and 6 weeks after surgery using the Celeris Diagnosys system (Diagnosys LLC, Lowell, MA). Anesthesia was administered, and the mice were positioned on a heating table to maintain body temperature. Pupillary dilation was achieved using 1% tropicamide. Room illumination was maintained with dim red light. Gonak gel (Akorn, Inc., Lake Forest, IL) was applied to the cornea of each eye before electrode placement. ERG was recorded from the corneal surface using Diagnosys Celeris pattern stimulator electrodes while simultaneously presenting visual stimulation. Reference and ground electrodes are small stainless-steel needles inserted in the skin of the forehead area and the base of the tail, respectively. Prior to recording, electrode impedance was measured, ensuring electrodes had impedance values between 5-7 kΩ.

Horizontal contrast-reversing gratings were presented via Diagnosys Celeris pattern stimulator electrodes, with a visual stimulus contrast of 95%, luminance of 50 cd m⁻², spatial resolution of 0.052 cycles/degree, and a reversal frequency of 2.1 Hz. Each data trace captured lasted for 470 ms, including a 50 ms baseline prior to stimulus onset. The signal was sampled at a frequency of 2000 Hz. The average of two consecutive recordings of 300 traces each was taken to produce one reading. The pERG measurements included the mean amplitudes of the P1 (first positive peak) and N2 (second negative peak) components. The P1 component represents the initial positive deflection, originating from RGCs and outer retinal photoreceptors. The mean P1 amplitude was measured as the difference between the average lowest point of the N1 component and the average peak of the P1 component. The N2 component is the negative deflection following the P1 component, originating from the inner retina and reflecting RGC function. The mean N2 amplitude was calculated as the difference between the average peak of the P1 component and the average lowest point of the N2 component.

### Flash Visual Evoked Potential (VEP)

Flash VEP testing was performed on mice prior to surgery and at 2, 4, and 6 weeks post-viscobead injection using the Celeris Diagnosys system (Diagnosys LLC, Lowell, MA). Mice were dark-adapted overnight before the VEP recordings. Anesthesia was administered, and the mice were positioned on a heating table to maintain body temperature. Pupillary dilation was achieved using 1% tropicamide. Room illumination was maintained with dim red light. Gonak gel (Akorn, Inc., Lake Forest, IL) was applied to the cornea of each eye before electrode placement. We adhered to the standard protocols of the International Society for Clinical Electrophysiology of Vision (ISCEV). Prior to recording, electrode impedance was measured, ensuring recording electrodes had impedance values below 2 kΩ. White flash stimulation was delivered using the Diagnosys Celeris touch stimulator electrodes at an intensity of 1 cd s⁻¹ m⁻². Each stimulus lasted for 5 ms with an interval of 10 s. Each recorded trace had a duration of 320 ms, including a 20 ms pre-stimulus baseline. The signals were amplified 1,000 times with a bandpass filter set between 0.1 Hz and 300 Hz. Each waveform was averaged over 61 superimposed responses to enhance signal-to-noise ratio. Flash VEP inspection was performed using a two-channel recording method and measured at least three times consecutively.

### Immunohistochemistry

For immunostaining, the animals were euthanized with an overdose of anesthesia, and perfused transcardially with ice-cold PBS, followed by 4% paraformaldehyde (PFA, Sigma). Subsequently, the optic nerves were carefully dissected and postfixed in 4% PFA overnight at 4° C. To ensure cryoprotection, the tissues were immersed in a 30% sucrose solution in PBS for 48 hours. Samples were then embedded in Optimal Cutting Temperature compound (Tissue Tek) using dry ice and sectioned into 12 μm sections for the optic nerves. Dissected retinas were rinsed in PBS and subsequently blocked in a solution containing PBS, 1% Triton X-100, and 5% horse serum (whole-mount buffer) overnight at 4° C. Primary antibodies, diluted in the whole-mount buffer, were applied for 2-4 days at 4° C., followed by three washes with PBS (each lasting 10 minutes). Secondary antibodies, all diluted at a 1:1000 ratio in PBS, were applied and left overnight at 4° C. After five washes with PBS (each lasting 10 minutes), the retinas were mounted using Fluoromount-G (Southern Biotech, Cat. No. 0100-01). Confocal images were collected with Leica SP2, Zeiss 700 or Zeiss 710 confocal microscopes. Image stacking and quantitative analysis of RGC loss were processed by Fiji software or CellCount software.

### Data Analysis

Normality and variance similarity were assessed using Microsoft Excel and the R programming language before applying any parametric tests. If the criteria for parametric tests were not met, non-parametric tests were carried out. For single comparisons between two groups, a two-tailed Student's t-test was utilized. In cases of multiple groups, the data were analyzed using either one-way or two-way ANOVA, depending on the appropriate experimental design. Post hoc comparisons were performed only when statistical significance was observed in the primary measure, and Bonferroni's correction was used to adjust the p-values for multiple comparisons. Error bars in all figures represent the mean±S.E.M. The mice, which had varying litters, body weights, and sexes, were randomized and assigned to different treatment groups. No additional randomization methods were employed for the animal studies. Each experimental data value represents individual measurements or observations, and their descriptions are provided in the corresponding figure captions. All statistical analyses were conducted using GraphPad Prism 10. Significance was determined with P values less than 0.05, denoted as follows: *0.01≤p<0.05, **0.001≤p<0.01, ***0.0001≤p<0.001, ****p<0.0001, and “ns” for not significant. Detailed statistical information and measurements values for the figures are provided in the supplementary tables.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compounds or compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 1999, and “March's Advanced Organic Chemistry”, 5th Ed.: Smith, M. B. and March, J., John Wiley & Sons, New York: 2001, the entire contents of which are hereby incorporated by reference.

As used herein, the terms “comprises,” “comprising”, or “having” when used to define compositions and methods, are intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Applicant's disclosure is described herein in preferred embodiments with reference to the figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A bead comprising:
a core comprising polystyrene (PS) or latex; and
a shell encapsulating the core, wherein the shell comprises poly(lactic-co-glycolic acid) (PLGA), poly(glycolic acid) (PGA) or polylactic acid (PLA),
wherein
the core has a diameter in the range of about 1 μm to about 20 μm, and
the shell has a thickness in the range of about 1 μm to about 10 μm.

2. The bead of claim 1, wherein the core is non-degradable.

3. The bead of claim 1, wherein the core is PS.

4. The bead of claim 3, wherein the shell is PLGA.

5. The bead of claim 4, wherein
the PS is characterized by a MWₙ of about 5,000 to about 400,000; and
the PLGA is characterized by a MWₙ of about 1,000 to about 300,000.

6. The bead of claim 5, wherein
the PS is characterized by a MWₙ of about 35,000 to about 50,000; and
the PLGA is characterized by a MWₙ of about 25,000 to about 35,000.

7. The bead of claim 5, wherein the PLGA has about 5% to about 95% lactic acid (LA) and about 95% to about 5% glycolic acid (GA).

8. The bead of claim 7, wherein the PLGA has about 35% to about 65% LA and about 65% to about 35% GA.

9. The bead of claim 8, the core has a diameter in the range of about 1 μm to about 10 μm, and the shell has a thickness in the range of about 1 μm to about 10 μm.

10. The bead of claim 1, wherein the core is latex.

11. The bead of claim 1, wherein the shell is PGA.

12. A composition comprising a plurality of beads according to claim 1.

13. The composition of claim 12, wherein the plurality of beads is characterized by a heterogeneous size distribution of beads within the range of about 1 μm to about 50 μm.

14. (canceled)

15. An animal glaucoma model induced by the plurality of beads according to claim 12.

16. The animal model of claim 15, wherein the animal is a mouse.

17. The animal model of claim 15, characterized by an intraocular pressure (IOP) elevation of about 5 mm Hg to about 25 mm Hg.

18. The animal model of claim 17, wherein the IOP elevation is about 10 mm Hg to about 20 mm Hg.

19. A method for preparing an animal glaucoma model, comprising injecting to the anterior chamber of the animal's eyes a plurality of beads according to claim 12.

20. The method of claim 19, wherein the animal is a mouse.

21. A method for preparing PS/PLGA core-shell beads, comprising:
providing a first emulsion of PS in an organic solvent;
proving a second aqueous solution of poly(vinyl alcohol) (PVA);
mixing the first emulsion with the second solution to form a double emulsion;
evaporating the organic solvent and removing the aqueous solvent to obtain PS microspheres;
adding the PS microspheres to a PLGA solution, followed addition of the aqueous PVA solution and emulsification of the resulting solution; and
isolating the prepared PS/PLGA microspheres.

22-24. (canceled)
